(19) United States
(12) Patent Application Publication
(10) Pub. No.: US 2014/0335046 A1
(43) Pub. Date: Nov. 13, 2014

(54) Compositions, Methods and Systems for Regenerative Cosmetics

(71) Applicant: Robert G. Matheny, Norcross, GA (US)

(72) Inventor: Robert G. Matheny, Norcross, GA (US)

(21) Appl. No.: 14/031,321

(22) Filed: Sep. 19, 2013

Related U.S. Application Data
(60) Provisional application No. 61/822,486, filed on May 13, 2013.

(51) Int. Cl. A61K 8/98; A61Q 19/00

(52) U.S. Cl. CPC A61K 8/981; A61Q 19/00
USPC 424/85.1; 424/130.1; 424/93.7; 424/93.71; 424/94.63; 424/551; 424/557; 424/558; 424/569; 424/583

(57) ABSTRACT

A method of inducing or promoting hair growth on the scalp of a subject, comprising the steps of (i) providing an ECM composition including at least one ECM material, (ii) administering inciting event means to a target skin location on the subject to induce an inciting event, and (iii) administering a therapeutically effective amount of said ECM composition to the target skin location. In some embodiments, the ECM composition includes at least one additional biologically active agent.

### CROSS-REFERENCES TO RELATED APPLICATIONS

This application claims the benefit of U.S. Application No. 61/822,486, filed on May 3, 2013.

### FIELD OF THE INVENTION

The present invention relates to compositions, methods, and systems for regenerative cosmetic treatments and procedures. More particularly, the present invention relates to extracellular matrix (ECM) compositions, and methods and systems using same, in cosmetic products, treatments, and procedures.

### BACKGROUND OF THE INVENTION

As is well known in the art, many compositions, methods and systems exist for cosmetic procedures such as wrinkle removal, tightening of the skin, improvement of appearance of skin, etc. Many of these cosmetic procedures can be used to treat an underlying condition or disease, or scars resulting therefrom.

Traditional treatments and procedures for cosmetics range from more conservative modalities, such as powders and lotions that may or may not be medicated, to more in-depth modalities as Botox® injections, and surgical reconstructions.

Notwithstanding the abundance of research activities and development of cosmetic treatments, unfortunately, there is yet no therapeutically effective method capable of fundamentally regenerating skin or its constituents. Alternative methods, which are currently available by modern medical techniques, include, for example, surgical procedures, such as skin transplantation and facial plastic surgery, and various pharmacological (or drug) therapies.

### SUMMARY OF THE INVENTION

The present invention is directed to administration of extracellular matrix (ECM) compositions and methods and systems employing same in cosmetic treatments and procedures.

In a preferred embodiment, the ECM compositions include at least one ECM material.

In a preferred embodiment of the invention, the ECM material comprises mammalian extracellular matrix tissue selected from the group comprising small intestine submucosa (SIS), urinary bladder submucosa (UBS), stomach submucosa (SS), central nervous system tissue, epithelium of mesodermal origin, i.e. mesothelial tissue, dermal extracellular matrix, subcutaneous extracellular matrix, gastrointestinal extracellular matrix, i.e. large and small intestines, tissue surrounding growing bone, placental extracellular matrix, ornamentum extracellular matrix, cardiac extracellular matrix, e.g., pericardium and/or myocardium, kidney extracellular matrix, pancreas extracellular matrix, lung extracellular matrix, and combinations thereof.

In some embodiments of the invention, the ECM compositions include at least one supplemental biologically active agent or composition, i.e. an agent that induces or modulates a physiological or biological process, or cellular activity, e.g. promotes tissue remodeling.

In some embodiments of the invention, the biologically active agent comprises a growth factor selected from the group comprising a platelet derived growth factor (PDGF), epidermal growth factor (EGF), transforming growth factor-α (TGF-α), transforming growth factor-β (TGF-β), fibroblast growth factor-2 (FGF-2), basic fibroblast growth factor (bFGF), vascular epithelial growth factor (VEGF), hepatocyte growth factor (HGF), insulin-like growth factor (IGF), nerve growth factor (NGF), platelet derived growth factor (PDGF), tumor necrosis factor-α (TNA-α), and placental growth factor (PLGF).

In some embodiments, the biologically active agent comprises a cell selected from the group comprising a human embryonic stem cell, fetal cardiomyocyte, myofibroblast, mesenchymal stem cell, autotransplanted expanded cardiomyocytes, adipocyte, totipotent cell, pluripotent cell, blood stem cell, myoblast, adult stem cell, bone marrow cell, mesenchymal cell, embryonic stem cell, parenchymal cell, epithelial cell, endothelial cell, mesothelial cell, fibroblast, osteoblast, chondrocyte, exogenous cell, endogenous cell, hematopoietic stem cell, bone-marrow derived progenitor cell, myocardial cell, skeletal cell, fetal cell, undifferentiated cell, multi-potent progenitor cell, unipotent progenitor cell, monocyte, cardiac myoblast, skeletal myoblast, macrophage, capillary endothelial cell, xenogenic cell, allogenic cell and post-natal stem cell.

In some embodiments, the biologically active agent is selected from the group comprising collagen (types I-V), proteoglycans, glycosaminoglycans (GAGs), glycoproteins, growth factors, cytokines, cell-surface associated proteins, cell adhesion molecules (CAM), angiogenic growth factors, endothelial ligands, matrikines, cadherins, immuoglobins, fibril collagens, non-fibrallar collagens, basement membrane collagens, multiplexins, small-leucine rich proteoglycans, decorins, biglycans, fibromodulins, keratocans, lumicans, epiphycans, heparin sulfate proteoglycans, perlecans, agrins, testicans, syndecans, glypicans, serglycins, selectins, lecticans, aggrecans, versicans, neurocans, brevicans, cytoplasmic domain-44 (CD-44), macrophage stimulating factors, amyloid precursor proteins, heparins, chondroitin sulfate B (dermatan sulfate), chondroitin sulfate A, heparin sulfates, hyaluronic acids, fibronectins, tenascins, elastins, fibrillins, laminins, nidogen/enactins, fibulin I, finulin II, integrins, transmembrane molecules, thrombospondins, ostepontins, and angiotensin converting enzymes (ACE).

In some embodiments of the invention, the biologically active agent comprises a pharmacological agent or composition (or drug), i.e. an agent or composition that is capable of producing a desired biological effect in vivo, e.g., stimulation or suppression of an immune response, etc.

In some embodiments, the pharmacological agent comprises an anti-inflammatory agent.

In some embodiments, the pharmacological agent comprises a statin, i.e. a HMG-CoA reductase inhibitor.

In some embodiments of the invention, the ECM material comprises a decellularized ECM material.

In a preferred embodiment, the ECM material is decellularized via the use of supercritical carbon dioxide and a rapid vessel depressurization step.

In accordance with another embodiment of the invention, there is provided a system for delivering a fluidized ECM composition to a region on a body wherein cosmetic treatment is desired.

In some embodiments, the system comprises an ECM composition and associated injection means that allows the ECM composition to be administered to a desired region on a body and/or depth within the region. For example, lip augmentation commonly consists of procedures designed to improve the appearance of the lips by increasing their fullness through enlargement (i.e. injection of a bulking agent or filler). This procedure can include intradermal administration of an ECM composition either concomitantly with or in place of standard compounds such as collagen, hyaluronic acid, and/or other compounds common in the art.

Another example includes elimination or reduction of the appearance of wrinkles via intradermal injections with an ECM composition of the invention. Common wrinkles or aging lines that are treated include nasolabial folds (nose to mouth lines), melomental folds (sad mouth corners), “crow's feet”, and forehead wrinkles amongst others. It is anticipated that the regenerative properties of the ECM within the ECM composition will impart increased tightness on the treated area as well as a “filler” effect wherein the increased volume of the area increases the tension of the skin thereby reducing the appearance of wrinkles.

In some embodiments, the ECM composition is presented as a powder and applied directly to the skin in regions wherein cosmetic alterations are desired. The powdered ECM composition can partially consist of decellularized extracellular matrix grafts that are ground down to particulates between approximately 5 μm and 500 μm in size. The ECM composition can also contain additives to alter coloration of skin, such as dihydroxyacetone (DHA). In accordance with another feature of the invention, the ECM composition can further be administered concomitantly with cosmetics that alter appearance.

In accordance with another feature of the invention, the ECM composition can be administered concomitantly with cosmetics, such as, for example, foundation(s) that contain ingredients, such as coverage pigments consisting of zinc oxide and/or titanium oxide, mica (sericite), iron oxide, tin oxide, and/or other compounds common in the art.

In some embodiments, the ECM composition is presented as a lotion or cream applied directly to the skin in regions wherein cosmetic alterations are desired. The ECM composition can consist of aqueous and oily phases, an emulgent to prevent separation, particulate ECM, and potentially, additional compounds or biologically active agents. In one embodiment, the ECM particulates are between approximately 5 μm and 500 μm.

In some embodiments, the ECM composition is administered to a wound or site of trauma to reduce the appearance of scarring as the wound or other trauma heals. The ECM composition can contain topical antibiotics, such as erythromycin, bacitracin, and/or neomycin to protect the healing wound from infection while also imparting the regenerative properties associated with ECM. For example, a cosmetic procedure to removal skin growths can be treated with an ECM composition post operation to increase the speed of recovery, increase resistance to infection, and/or reduce the potential for fibrosis.

In some embodiments, the ECM composition is administered after a skin procedure, such as a chemical peel. A chemical peel is administered to a desired area where dead skin is sloughed off and eventually peeled off. The depth to which the chemical peel reaches within the skin depends on the chemical being administered. The ECM composition can be used for treatments ranging from lighter chemical peels, such as alpha hydroxy acid peels that just lightly remove the top layer of dead skin cells of the epidermis to more profound treatments, such as a phenol peel that remove skin as deep down as the lower dermis and subcutis. The administration of the ECM composition allows for increased speed of recovery, increased resistance to infection, and reduced potential for fibrosis as well as increased tightness of the surface of the skin due to generation of a epidermis and/or dermis.

In some embodiments, the ECM composition is administered after the patient undergoes a skin treatment procedure wherein a laser resurfaces the skin, removing fine lines, wrinkles, scars. Numerous lasers are typically employed, but the majority of the procedures are completed using either a carbon dioxide (CO₂) or erbium laser. The ECM composition can be administered shortly after the laser treatment and can accelerate healing time as well as compliment the removal of lesions, wrinkles, and/or scars due to the regenerative nature of the ECM.

### BRIEF DESCRIPTION OF THE DRAWINGS

Further features and advantages will become apparent from the following and more particular description of the preferred embodiments of the invention, as illustrated in the accompanying drawings, and in which like referenced characters generally refer to the same parts or elements throughout the views, and in which:

FIG. 1 is a diagram illustrating the layers of human skin.

### DETAILED DESCRIPTION OF THE PREFERRED EMBODIMENT

Before describing the present invention in detail, it is to be understood that this invention is not limited to particularly exemplified compositions, apparatus, systems, structures or methods as such may, of course, vary. Thus, although a number of compositions, apparatus, systems and methods similar or equivalent to those described herein can be used in the practice of the present invention, the preferred compositions, apparatus, systems, structures and methods are described herein.

It is also to be understood that the terminology used herein is for the purpose of describing particular embodiments of the invention only and is not intended to be limiting.

Unless defined otherwise, all technical and scientific terms used herein have the same meaning as commonly understood by one having ordinary skill in the art to which the invention pertains.

Further, all publications, patents and patent applications cited herein, whether supra or infra, are hereby incorporated by reference in their entirety.

As used in this specification and the appended claims, the singular forms “a,” “an” and “the” include plural referents unless the content clearly dictates otherwise. Thus, for example, reference to “a pharmacological agent” includes two or more such agents and the like.

Further, ranges can be expressed herein as from “about” or “approximately” one particular value, and/or to “about” or “approximately” another particular value. When such a range is expressed, another embodiment includes from the one particular value and/or to the other particular value. Similarly, when values are expressed as approximations, by use of the antecedent “about” or “approximately”, it will be understood that the particular value forms another embodiment. It will be further understood that the endpoints of each of the ranges are significant both in relation to the other endpoint, and independently of the other endpoint.

It is also understood that there are a number of values disclosed herein, and that each value is also herein disclosed as “about” or “approximately” that particular value in addition to the value itself. For example, if the value “10” is disclosed, then “approximately 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed then “less than or equal to 10” as well as “greater than or equal to 10” is also disclosed.

### DEFINITIONS

The term“inciting event”, as used herein, means and includes an event or action that causes cellular damage or injury and/or induces cellular migration or a cascade of cytokines and/or growth factors and/or other molecules, and/or other cellular activities that are associated with tissue remodeling or wound repair.

The terms “extracellular matrix”, “ECM” and “ECM material” are used interchangeably herein, and mean and include a collagen-rich substance that is found in between cells in mammalian tissue, and any material processed therefrom, e.g. decellularized ECM. According to the invention, the ECM material can be derived from a variety of mammalian tissue sources, including, without limitation, small intestine submucosa (SIS), urinary bladder submucosa (UBS), stomach submucosa (SS), central nervous system tissue, epithelium of mesodermal origin, i.e. mesothelial tissue, dermal extracellular matrix, subcutaneous extracellular matrix, gastrointestinal extracellular matrix, i.e. large and small intestines, tissue surrounding growing bone, placental extracellular matrix, ornamentum extracellular matrix, cardiac extracellular matrix, e.g., pericardium and/or myocardium, kidney extracellular matrix, pancreas extracellular matrix, lung extracellular matrix, and combinations thereof. The ECM material can also comprise collagen from mammalian sources.

The terms “urinary bladder submucosa (UBS)”, “small intestine submucosa (SIS)” and “stomach submucosa (SS)” also mean and include any UBS and/or SIS and/or SS material that includes the tunica mucosa (which includes the transitional epithelial layer and the tunica propria), submucosal layer, one or more layers of muscularis, and adventitia (a loose connective tissue layer) associated therewith.

The ECM material can also be derived from basement membrane of mammalian tissue/organs, including, without limitation, urinary basement membrane (UBM), liver basement membrane (LBM), and amnion, chorion, allograft pericardium, allograft acellular dermis, amniotic membrane, Wharton's jelly, and combinations thereof.

Additional sources of mammalian basement membrane include, without limitation, spleen, lymph nodes, salivary glands, prostate, pancreas and other secreting glands.

The ECM material can also be derived from other sources, including, without limitation, collagen from plant sources and synthesized extracellular matrices, i.e. cell cultures.

The term “acellular”, as used herein, means extracellular matrix compositions that are at least 80% decellularized, such that the extracellular matrix composition is 80% without cells and/or cellular remnants. In some exemplary aspects described herein, the term “acellular” can thus refer to extracellular matrix compositions that are at least 90% decellularized such that the extracellular matrix composition is at least 90% without cells and/or cellular remnants.

Thus, as used herein, the term “acellular” can refer to extracellular matrix compositions that are decellularized at levels of 80%, 81%, 82%, 83%, 84%, 85%, 86%, 87%, 88%, 89%, 90%, 91%, 92%, 93%, 94%, 95%, 96%, 97%, 98%, 99%, 100%, and any percentages falling between these values.

The term “angiogenesis”, as used herein, means a physiologic process involving the growth of new blood vessels from pre-existing blood vessels.

The term “neovascularization”, as used herein, means and includes the formation of functional vascular networks that can be perfused by blood or blood components. Neovascularization includes angiogenesis, budding angiogenesis, intussuceptive angiogenesis, sprouting angiogenesis, therapeutic angiogenesis and vasculogenesis.

The terms “biologically active agent” and “biologically active composition” are used interchangeably herein, and mean and include agent that induces or modulates a physiological or biological process; or cellular activity, e.g. induces cellular migration or differentiation.

The terms “biologically active agent” and “biologically active composition” thus mean and include, without limitation, the following growth factors: a platelet derived growth factor (PDGF), epidermal growth factor (EGF), transforming growth factor-α (TGF-α), transforming growth factor-β (TGF-β), fibroblast growth factor-2 (FGF-2), basic fibroblast growth factor (bFGF), vascular epithelial growth factor (VEGF), hepatocyte growth factor (HGF), insulin-like growth factor (IGF), nerve growth factor (NGF), platelet derived growth factor (PDGF), tumor necrosis factor-α (TNA-α), and placental growth factor (PLGF).

The terms “biologically active agent” and “biologically active composition” also mean and include, without limitation, human embryonic stem cells, fetal cardiomyocytes, myofibroblasts, mesenchymal stem cells, autotransplanted expanded cardiomyocytes, adipocytes, totipotent cells, pluripotent cells, blood stem cells, myoblasts, adult stem cells, bone marrow cells, mesenchymal cells, embryonic stem cells, parenchymal cells, epithelial cells, endothelial cells, mesothelial cells, fibroblasts, osteoblasts, chondrocytes, exogenous cells, endogenous cells, stem cells, hematopoietic stem cells, bone-marrow derived progenitor cells, myocardial cells, skeletal cells, fetal cells, undifferentiated cells, multi-potent progenitor cells, unipotent progenitor cells, monocytes, cardiac myoblasts, skeletal myoblasts, macrophages, capillary endothelial cells, xenogenic cells, allogenic cells, and post-natal stem cells.

The terms “biologically active agent” and “biologically active composition” also mean and include, without limitation, the following biologically active agents (referred to interchangeably herein as a “protein”, “peptide” and “polypeptide”): collagen (types I-V), proteoglycans, glycosaminoglycans (GAGs), glycoproteins, growth factors, cytokines, cell-surface associated proteins, cell adhesion molecules (CAM), angiogenic growth factors, endothelial ligands, matrikines, cadherins, immunoglobin, fibril collagens, non-fibrillar collagens, basement membrane collagens, multiplexins, small-leucine rich proteoglycans, decorins, biglycans, fibromodulins, keratocans, lumicans, epiphycans, heparin sulfate proteoglycans, perlecans, agrins, testicans, syndecans, glypicans, serglycins, selectins, lecticans, aggrecans, versicans, neurocans, brevicans, cytoplasmic domain-44 (CD-44), macrophage stimulating factors, amyloid precursor proteins, heparins, chondroitin sulfate B (dermatan sulfate), chondroitin sulfate A, heparin sulfates, hyaluronic acids, fibronectins, tenascins, elastins, fibrillins, laminins, nidogen/enactins, fibulin I, finulin II, integrins, transmembrane molecules, thrombospondins, ostepontins, and angiotensin converting enzymes (ACE).

The terms “pharmacological agent”, “active agent”, “drug” and “active agent formulation” are used interchangeably herein, and mean and include an agent, drug, compound, composition of matter or mixture thereof, including its formulation, which provides some therapeutic, often beneficial, effect. This includes any physiologically or pharmacologically active substance that produces a localized or systemic effect or effects in animals, including warm blooded mammals, humans and primates; avians; domestic household or farm animals, such as cats, dogs, sheep, goats, cattle, horses and pigs; laboratory animals, such as mice, rats and guinea pigs; fish; reptiles; zoo and wild animals; and the like.

The terms “pharmacological agent”, “active agent”, “drug” and “active agent formulation” thus mean and include, without limitation, antibiotics, anti-arrhythmic agents, anti-viral agents, analgesics, steroidal anti-inflammatories, non-steroidal anti-inflammatories, anti-neoplastics, anti-spasmodics, modulators of cell-extracellular matrix interactions, proteins, hormones, growth factors, matrix metalloproteinases (MMPS), enzymes and enzyme inhibitors, anticoagulants and/or antithrombic agents, DNA, RNA, modified DNA and RNA, NSAIDs, inhibitors of DNA, RNA or protein synthesis, polypeptides, oligonucleotides, polynucleotides, nucleoproteins, compounds modulating cell migration, compounds modulating proliferation and growth of tissue, and vasodilating agents.

The terms “pharmacological agent”, “active agent”, “drug” and “active agent formulation” thus include, without limitation, atropine, tropicamide, dexamethasone, dexamethasone phosphate, betamethasone, betamethasone phosphate, prednisolone, triamcinolone, triamcinolone acetonide, fluocinolone acetonide, anecortave acetate, budesonide, cyclosporine, FK-506, rapamycin, ruboxistaurin, midostaurin, flurbiprofen, suprofen, ketoprofen, diclofenac, ketorolac, nepafenac, lidocaine, neomycin, polymyxin b, bacitracin, gramicidin, gentamicin, oyxtetracycline, ciprofloxacin, ofloxacin, tobramycin, amikacin, vancomycin, cefazolin, ticarcillin, chloramphenicol, miconazole, itraconazole, trifluridine, vidarabine, ganciclovir, acyclovir, cidofovir, ara-amp, foscarnet, idoxuridine, adefovir dipivoxil, methotrexate, carboplatin, phenylephrine, epinephrine, dipivefrin, timolol, 6-hydroxydopamine, betaxolol, pilocarpine, carbachol, physostigmine, demecarium, dorzolamide, brinzolamide, latanoprost, sodium hyaluronate, insulin, verteporfin, pegaptanib, ranibizumab, and other antibodies, antineoplastics, anti VGEFs, ciliary neurotropic factor, brain-derived neurotropic factor, bFGF, Caspase-1 inhibitors, Caspase-3 inhibitors, α-Adrenoceptors agonists, NMDA antagonists, Glial cell line-derived neurotropic factors (GDNF), pigment epithelium-derived factor (PEDF), and NT-3, NT-4, NGF, IGF-2.

The terms “pharmacological agent”, “active agent”, “drug” and “active agent formulation” further mean and include, without limitation, the following antibiotics: aminoglycosides, cephalosporins, chloramphenicol, clindamycin, erythromycins, fluoroquinolones, macrolides, azolides, metronidazole, penicillins, tetracyclines, trimethoprim-sulfamethoxazole and vancomycin.

The terms “pharmacological agent”, “active agent”, “drug” and “active agent formulation” further include, without limitation, the following steroids: andranes (e.g., testosterone), cholestanes, cholic acids, corticosteroids (e.g., dexamethasone), estraenes (e.g., estradiol) and pregnanes (e.g., progesterone).

The terms “pharmacological agent”, “active agent”, “drug” and “active agent formulation” can further include one or more classes of topical or local anesthetics, including, without limitation, esters, such as benzocaine, chloroprocaine, cocaine, cyclomethycaine, dimethocaine/larocaine, piperocaine, propoxycaine, procaine/novacaine, proparacaine, and tetracaine/amethocaine. Local anesthetics can also include, without limitation, amides, such as articaine, bupivacaine, cinchocaine/dibucaine, etidocaine, levobupivacaine, lidocaine/lignocaine, mepivacaine, prilocaine, ropivacaine, and trimecaine. Local anesthetics can further include combinations of the above from either amides or esters.

The terms “pharmacological agent”, “active agent”, “drug” and “active agent formulation” can further include one or more classes of cytotoxic anti-neoplastic agents or chemotherapy agents, including, without limitation, alkylating agents, cisplatin, carboplatin, oxaliplatin, mechlorethamine, cyclophosphamide, chlorambucil, and Ifosfamide. Chemotherapy agents can also include, without limitation, antimetabolites, such as purine analogues, pyrimidine analogues and antifolates, plant alkaloids, such as vincristine, vinblastine, vinorelbine, vindesine, podophyllotoxin, etoposide and teniposide, taxanes, such as paclitaxel and docetaxel, topoisomerase inhibitors, such as irinotecan, topotecan, amsacrine, etoposide, etoposide phosphate and teniposide, cytotoxic antibiotics, such as actinomyocin, bleomycin, plicamycin, mytomycin and anthracyclines, such as doxorubicin, daunorubicin, valrubicin, idarubicin, epirubicin, and antibody treatments, such as abciximab, adamlimumab, alamtuzumab, basiliximab, belimumab, bevacizumab, brentuximab vedotin, canakinumab, cetuximab, certolizumab pego, daclizumab, denosumab, eculizumab, efalizumab, gemtuzumab, golimumab, ibritumomab tiuxetan, infliximab, ipilimumab, muromonab-CD3, natalizumab, ofatumumab, omalizumab, palivizumab, panitumumab, ranibizumab, rituximab, tocilizumab (atlizumab), tositumomab and trastuzumab.

The term “emulsion”, as used herein, means a mixture in which a first ECM material is dispersed within a second ECM material, with the first ECM material being immiscible with the second ECM material. The “emulsions” described herein can refer to either oil-in-water type emulsions or water-in-oil type emulsions.

The term “suspension”, as used herein, means a mixture in which a solid ECM material, such as, for example and without limitation, particulate ECM, is dispersed (suspended) in a fluid ECM material, such as, for example and without limitation, ECM gel or ECM liquid.

The term “biocompatible”, as used herein, means a device or material that is substantially non-toxic in an in vivo environment, and is not substantially rejected by a recipient's physiological system, i.e. non-antige.

The terms “anti-inflammatory” and “anti-inflammatory agent” are also used interchangeably herein, and mean and include a “pharmacological agent” and/or “active agent formulation”, which, when a therapeutically effective amount is administered to a subject, prevents or treats bodily tissue inflammation i.e. the protective tissue response to injury or destruction of tissues, which serves to destroy, dilute, or wall off both the injurious agent and the injured tissues.

Anti-inflammatory agents thus include, without limitation, alclofenac, alclometasone dipropionate, algestone acetonide, alpha amylase, amcinafal, amcinafide, amfenac sodium, amiprilose hydrochloride, anakinra, anirolac, anitrazafen, apazone, balsalazide disodium, bendazac, benoxaprofen, benzydamine hydrochloride, bromelains, broperamole, budesonide, carprofen, cicloprofen, cintazone, cliprofen, clobetasol propionate, clobetasone butyrate, clopirac, cloticasone propionate, cormethasone acetate, cortodoxone, decanoate, deflazacort, delatestryl, depo-testosterone, desonide, desoximetasone, dexamethasone dipropionate, diclofenac potassium, diclofenac sodium, diflorasone diacetate, diflumidone sodium, diflunisal, difluprednate, diftalone, dimethyl sulfoxide, drocinonide, endrysone, enlimomab, enolicam sodium, epirizole, etodolac, etofenamate, felbinac, fenamole, fenbufen, fenclofenac, fenclorac, fendosal, fenpipalone, fentiazac, flazalone, fluazacort, flufenamic acid, flumizole, flunisolide acetate, flunixin, flunixin meglumine, fluocortin butyl, fluorometholone acetate, fluquazone, flurbiprofen, fluretofen, fluticasone propionate, furaprofen, furobufen, halcinonide, halobetasol propionate, halopredone acetate, ibufenac, ibuprofen, ibuprofen aluminum, ibuprofen piconol, ilonidap, indomethacin, indomethacin sodium, indoprofen, indoxole, intrazole, isoflupredone acetate, isoxepac, isoxicam, ketoprofen, lofemizole hydrochloride, lomoxicam, loteprednol etabonate, meclofenamate sodium, meclofenamic acid, meclorisone dibutyrate, mefenamic acid, mesalamine, meseclazone, mesterolone, methandrostenolone, methenolone, methenolone acetate, methylprednisolone suleptanate, momiflumate, nabumetone, nandrolone, naproxen, naproxen sodium, naproxol, nimazone, olsalazine sodium, orgotein, orpanoxin, oxandrolane, oxaprozin, oxyphenbutazone, oxymetholone, paranyline hydrochloride, pentosan polysulfate sodium, phenbutazone sodium glycerate, pirfenidone, piroxicam, piroxicam cinnamate, piroxicam olamine, pirprofen, prednazate, prifelone, prodolic acid, proquazone, proxazole, proxazole citrate, rimexolone, romazarit, salcolex, salnacedin, salsalate, sanguinarium chloride, seclazone, sennetacin, stanozolol, sudoxicam, sulindac, suprofen, talmetacin, talniflumate, talosalate, tebufelone, tenidap, tenidap sodium, tenoxicam, tesicam, tesimide, testosterone, testosterone blends, tetrydamine, tiopinac, tixocortol pivalate, tolmetin, tolmetin sodium, triclonide, triflumidate, zidometacin, and zomepirac sodium.

The term “ECM composition”, as used herein, thus means and includes an ECM material in combination with a “biologically active agent” and/or a “pharmacological agent” and/or any additional agent or component identified herein.

The term “therapeutically effective”, as used herein, means that the amount of the ECM composition administered to a subject is of sufficient quantity to ameliorate one or more causes, symptoms, or sequelae of a disease or tissue or skin disorder. Such amelioration only requires a reduction or alteration, not necessarily elimination, of the cause, symptom, or sequelae of the disease or disorder.

The terms “delivering,” “delivery,” and “deliver” are used interchangeably herein, and mean the application of an ECM composition of the invention to a site on a subject, wherein the ECM composition imparts its remodeling and therapeutic properties.

The terms “prevent” and “preventing” are used interchangeably herein, and mean and include reducing the frequency or severity of a disease, pathological condition, disease or tissue or skin disorder. The terms do not require an absolute preclusion of the disease or condition. Rather, the terms include decreasing the chance for disease occurrence.

The terms “treat” and “treatment” are used interchangeably herein, and mean and include medical management of a patient with the intent to ameliorate one or more causes, symptoms, or cure, ameliorate, stabilize, or prevent a disease, pathological condition, or tissue or skin disorder. The terms include “active treatment”, i.e. treatment directed specifically toward the improvement of a disease, pathological condition, or tissue or skin disorder, and “causal treatment”, i.e. treatment directed toward removal of the cause of the associated disease, pathological condition, or disorder.

The terms “treat” and “treatment” further include “palliative treatment”, i.e. treatment designed for the relief of symptoms rather than the curing of the disease, pathological condition, or disorder, “preventative treatment”, i.e. treatment directed to minimizing or partially or completely inhibiting the development of the associated disease, pathological condition, or disorder, and “supportive treatment”, i.e. treatment employed to supplement another specific therapy directed toward the improvement of the associated disease, pathological condition, or disorder.

The terms “optional” and “optionally” mean that the subsequently described event, circumstance, or material may or may not occur or be present, and that the description includes instances where the event, circumstance, or material occurs or is present and instances where it does not occur or is not present.

The term “comprise” and variations of the term, such as “comprising” and “comprises,” means “including, but not limited to” and is not intended to exclude, for example, other additives, components, integers or steps.

The terms “subject”, “patient” and “recipient” are used interchangeably herein, and mean and include any warm blooded mammal.

The following disclosure is provided to further explain in an enabling fashion the best modes of performing one or more embodiments of the present invention. The disclosure is further offered to enhance an understanding and appreciation for the inventive principles and advantages thereof, rather than to limit in any manner the invention. The invention is defined solely by the appended claims including any amendments made during the pendency of this application and all equivalents of those claims as issued.

As discussed above, the present invention is directed to extracellular matrix (ECM) compositions, and methods and systems using same, in cosmetic procedures in an effort to alter appearance. As will readily be appreciated by one having ordinary skill in the art, the present invention substantially reduces or eliminates the disadvantages and drawbacks associated with prior art methods, compositions, and systems for cosmetic procedures.

In a preferred embodiment of the invention, the ECM compositions include at least one ECM material.

In a preferred embodiment, the ECM compositions comprise sterilized, acellular ECM compositions that are preferably formed by contemporaneously sterilizing and decellularizing an isolated ECM material.

Sterilized, acellular ECM compositions and methods for making same are set forth in Co-Pending application Ser. Nos. 13/480,140, 12/707,427, 13/480,205, and Ser. No. 11/747,028; which are incorporated by reference herein in their entirety.

In addition to decellularizing the ECM material as described herein, the rapid depressurization of the ECM material also can be used to incorporate desired sterilants and supplemental biologically active agents into the ECM material.

In exemplary aspects, the sterilized acellular ECM composition can comprise any known ECM component or material, including, for example and without limitation, mucosal layers and components, submucosal layers and components, muscularis layers and components, dermis, and/or basement membrane layers and components.

According to the invention, the ECM material can be derived from various mammalian tissue sources and methods for preparing same, such as disclosed in U.S. Pat. Nos. 7,550,004, 7,244,444, 6,379,710, 6,358,284, 6,206,931, 5,733,337 and 4,902,508 and U.S. application Ser. No. 12/707,427; which are incorporated by reference herein in their entirety. The mammalian tissue sources include, without limitation, small intestine submucosa (SIS), urinary bladder submucosa (UBS), stomach submucosa (SS), central nervous system tissue, epithelium of mesodermal origin, i.e. mesothelial tissue, dermal extracellular matrix, subcutaneous extracellular matrix, gastrointestinal extracellular matrix, i.e. large and small intestines, tissue surrounding growing bone, placental extracellular matrix, ornamentum extracellular matrix, cardiac extracellular matrix, e.g., pericardium and/or myocardium, kidney extracellular matrix, pancreas extracellular matrix, lung extracellular matrix, and combinations thereof. The ECM material can also comprise collagen from mammalian sources.

In a preferred embodiment of the invention, the ECM composition comprises mesothelial tissue.

As stated above, in some embodiments of the invention, the ECM composition (or material thereof) includes at least one additional biologically active agent or composition, i.e. an agent that induces or modulates a physiological or biological process, or cellular activity, e.g., induces proliferation, and/or growth and/or regeneration of tissue.

Suitable biologically active agents include any of the aforementioned biologically active agents, including, without limitation, the aforementioned cells and proteins.

In some embodiments, ECM composition includes at least one pharmacological agent or composition (or drug), i.e. an agent or composition that is capable of producing a desired biological effect in vivo, e.g., stimulation or suppression of apoptosis, stimulation or suppression of an immune response, etc.

Suitable pharmacological agents and compositions include any of the aforementioned agents, including, without limitation, antibiotics, anti-viral agents, analgesics, steroidal anti-inflammatories, non-steroidal anti-inflammatories, anti-neoplastics, anti-spasmodics, modulators of cell-extracellular matrix interactions, proteins, hormones, enzymes and enzyme inhibitors, anticoagulants and/or antithrombic agents, DNA, RNA, modified DNA and RNA, NSAIDs, inhibitors of DNA, RNA or protein synthesis, polypeptides, oligonucleotides, polynucleotides, nucleoproteins, compounds modulating cell migration, compounds modulating proliferation and growth of tissue, and vasodilating agents.

In some embodiments of the invention, the pharmacological agent comprises an anti-inflammatory agent.

In some embodiments of the invention, the pharmacological agent comprises a statin, i.e. a HMG-CoA reductase inhibitor. According to the invention, suitable statins include, without limitation, atorvastatin (Lipitor®), cerivastatin, fluvastatin (Lescol®), lovastatin (Mevacor®, Altocor®, Altoprev®), mevastatin, pitavastatin (Livalo®, Pitava®), pravastatin (Pravachol®, Selektine®, Lipostat®), rosuvastatin (Crestor®), and simvastatin (Zocor®, Lipex®). Several actives comprising a combination of a statin and another agent, such as ezetimbe/simvastatin (Vytorin®), are also suitable.

Applicant has found that the noted statins exhibit numerous beneficial properties that provide several beneficial biochemical actions or activities. The properties and beneficial actions are set forth in Applicant's Co-Pending application Ser. No. 13/373,569, filed on Sep. 24, 2012 and Ser. No. 13/782,024, filed on Apr. 1, 2013; which are incorporated by reference herein in their entirety.

In some embodiments of the invention, the pharmacological agent comprises chitosan. As also set forth in detail in Co-Pending application Ser. No. 13/573,569, chitosan also exhibits numerous beneficial properties that provide several beneficial biochemical actions or activities.

It is further contemplated that a disclosed sterilized acellular ECM composition can comprise an ECM material obtained from ECM components or materials of one or more mammals including, for example and without limitation, humans, cows, pigs, dogs, sheep, cats, horses, rodents, and the like. Thus, it is contemplated that a disclosed sterilized, acellular ECM composition can comprise ECM components or materials from two or more of the same mammalian species, such as, for example and without limitation, two or more cows, two or more pigs, two or more dogs, or two or more sheep.

It is further contemplated that a disclosed sterilized acellular ECM composition can comprise ECM components or materials from two or more different mammalian species, such as, for example and without limitation, a pig and a cow, a pig and a dog, a pig and a sheep, or a cow and a sheep. It is still further contemplated that a disclosed sterilized, acellular ECM composition can comprise ECM components or materials obtained from a first tissue source, such as, for example and without limitation, SIS, from a first mammal, as well as ECM components or materials obtained from a second tissue source, such as, for example and without limitation, SS, from a second mammal.

In various aspects, a disclosed sterilized acellular ECM composition can be produced in any suitable shape, including, for example and without limitation, a substantially flat sheet, a deformable structure, a deformable or moldable structure that conforms to the shape region of administration or a structure whose borders follow the outline of the region of administration. The ECM composition can also be formed as a multi-laminate and/or substantially spherical structure.

It is contemplated that a disclosed sterilized acellular ECM composition can also be produced in any suitable form, including, for example and without limitation, a solid, liquid, gel, particulate, emulsion, or suspension form similar to cosmetic compounds or lotions.

In one exemplary aspect, it is contemplated that a disclosed sterilized acellular ECM composition can comprise an outer layer of solid ECM material that encloses an inner layer of liquid, particulate, emulsion, suspension, and/or gel ECM material.

In another exemplary aspect, it is contemplated that a disclosed sterilized acellular ECM composition can comprise one or more types of particulate ECM materials that are suspended within an ECM gel to form an ECM suspension. In this aspect, it is contemplated that the particulates within a disclosed ECM suspension can have a diameter ranging from about 5 μm to about 500 μm, with an average diameter ranging from about 100 μm to about 200 μm.

It is further contemplated that the percentage of gel within a disclosed ECM suspension can range from about 5% to about 50%, while the percentage of particulate within a disclosed ECM suspension can range from about 50% to about 95%.

In a further aspect, it is contemplated that a disclosed ECM suspension can comprise sterilized, decellularized ECM.

In exemplary aspects, the ECM gel of a disclosed ECM suspension can be a hydrolyzed ECM. In these aspects, it is contemplated that the ECM gel of a disclosed ECM suspension can comprise ECM that is greater than about 50% hydrolyzed, more preferably, greater than about 70% hydrolyzed, and, most preferably, greater than about 90% hydrolyzed.

In one exemplary aspect, the ECM gel of a disclosed ECM suspension can comprise ECM that is about 100% hydrolyzed.

It is still further contemplated that the ECM components of the suspension can comprise at least one of the following: glycoproteins, such as, for example and without limitation, fibronectin and laminan; glycosaminoglycans, such as, for example and without limitation, heparan, hyaluronic acid, and chondroitin sulfate; and growth factors, thereby providing additional bioavailability for native cellular components.

It is contemplated that the ECM components of the suspension can provide a structural and biochemical microenvironment that promotes cell growth and stem cell attraction following administration of a disclosed ECM composition.

It is further contemplated that the ECM gel of a disclosed ECM suspension can function as a bulking agent that preserves a desired biomechanical environment until the cells of the subject can begin producing their own ECM.

It is further contemplated that the ECM gel of a disclosed ECM suspension can function as a tension inducing agent after implantation to reduce the appearance of wrinkles on the surface of the skin.

It is still further contemplated that the desired biomechanical environment that is preserved by the ECM gel can substantially correspond to a biomechanical environment in native tissue.

Thus, it is contemplated that the ECM gel of a disclosed ECM suspension can have an elastic modulus that is substantially equal to the elastic modulus of a target site within a subject.

In exemplary aspects, the elastic modulus of the ECM gel of a disclosed ECM suspension can range from about 5 kPa to about 50 kPa, and, more preferably, from about 10 kPa to about 15 kPa.

It is contemplated that one or more of the aforementioned supplemental biologically active agents can be incorporated into the ECM material to impart selected properties to the resulting sterilized acellular ECM composition.

In this aspect, it is contemplated that the one or more supplemental biologically active agents can be selected to replace or supplement components of the ECM material that are lost during processing of the ECM material. For example, and as described below, the one or more supplemental biologically active agents can comprise growth factors, cytokines, proteoglycans, glycosaminoglycans (GAGs), proteins, peptides, nucleic acids, small molecules, drugs, or cells.

It is further contemplated that the one or more supplemental biologically active agents can be selected to incorporate non-native components into the ECM material. For example, the one or more supplemental biologically active agents can comprise, for example and without limitation, growth factors for recruiting stem cells, angiogenic cytokines, and anti-inflammatory cytokines.

It is still further contemplated that the one or more supplemental biologically active agents can comprise one or more of the aforementioned pharmaceutical agents, such as statins, corticosteroids, non-steroidal anti-inflammatory drugs, anti-inflammatory compounds, anti-arrhythmic agents, and the like.

It is still further contemplated that the one or more supplemental biologically active agents can compromise cosmetic agents, such as additives to alter coloration of skin, such as dihydroxyacetone (DHA).

It is still further contemplated that the one or more supplemental biologically active agents can compromise cosmetic agents, such as foundation that contain ingredients such as coverage pigments consisting of zinc oxide and/or titanium oxide, mica (sericite), iron oxide, tin oxide, and/or other compounds common in the art.

It is still further contemplated that the one or more supplemental biologically active agents can comprise nanoparticles, such as, for example and without limitation, silver nanoparticles, gold nanoparticles, platinum nanoparticles, iridium nanoparticles, rhodium nanoparticles, palladium nanoparticles, copper nanoparticles, zinc nanoparticles, and other metallic nanoparticles.

It is still further contemplated that the one or more supplemental biologically active agents can comprise metallic compounds. In one exemplary aspect, the one or more additives can be selected to pharmaceutically suppress the immune response of a subject following implantation of the resulting ECM composition into the body of a subject.

In another aspect, the one or more supplemental biologically active agents can comprise one or more cytokines, including, for example and without limitation, stem cell factor (SCF), stromal cell-derived factor-1 (SDF-1), granulocyte macrophage colony-stimulating factor (GM-CSF), interferon gamma (IFN-gamma), Interleukin-3, Interleukin-4, Interleukin-10, Interleukin-13, Leukemia inhibitory factor (LIF), amphiregulin, thrombospondin 1, thrombospondin 2, thrombospondin 3, thrombospondin 4, thrombospondin 5, and angiotensin converting enzyme (ACE).

In an additional aspect, the one or more supplemental biologically active agents can comprise one or more proteoglycans, including, for example and without limitation, heparan sulfate proteoglycans, betaglycan, syndecan, decorin, aggrecan, biglycan, fibromodulin, keratocan, lumican, epiphycan, perlecan, agrin, testican, syndecan, glypican, serglycin, selectin, lectican, versican, neurocan, and brevican.

In a further aspect, the one or more supplemental biologically active agents can comprise one or more glycosaminoglycans, including, for example and without limitation, heparan sulfate, hyaluronic acid, heparin, chondroitin sulfate B (dermatan sulfate), and chondroitin sulfate A.

In a further aspect, the one or more supplemental biologically active agents can comprise statins, including, without limitation, cerevastatin, atorvastatin, fluvastatin, lovastatin, mevastatin, pitavastatin, pravastatin, rosuvastatin, and simvastatin.

In one optional aspect, in order to make the sterilized acellular ECM composition into a particulate form, the method can comprise cutting the ECM composition into pieces having desired lengths.

In another aspect, the method can optionally comprise freeze-drying the pieces of the ECM composition.

In an additional aspect, the method can optionally comprise grinding the frozen, hydrated pieces of the ECM composition and then passing the pieces of the ECM composition through a sizer screen until ECM particulate of a desired size is isolated.

In a further optional aspect, the method can comprise rehydrating the ECM particulate with sterile saline or other sterile, biocompatible fluid to form an ECM suspension, as described herein.

In a further aspect of the invention, prior to or simultaneously with delivery of an ECM composition of the invention, the target region of a subject is subjected to an inciting or stem cell attracting event, such as, without limitation, a laser treatment, chemical peel, electromagnetic radiation, mechanical agitation etc.

In one embodiment, a patient desiring cosmetic treatments for improvements in the appearance of facial skin undergoes a chemical peel consisting of administration of an acid to the desired site.

According to the invention, the ECM compositions can be delivered or administered to a subject via manual means, i.e. by hand, an applicator, injection means, or via a pressurized container, i.e. mist or spray application, or via an ECM delivery system of the invention. Upon administration of an ECM composition to a target region on a subject's body wherein treatment is desired, (i) new cells are generated, whereby a desired appearance can be achieved and (ii) the composition functions as a bulking agent while stimulation regeneration which can assist in achieving a desired appearance.

In a preferred embodiment, the administration of an ECM composition of the invention to the skin of a subject is preceded by or administrated simultaneously with an “inciting event”. As set forth above, the term “inciting event” means and includes an event or action that causes cellular damage or injury and/or induces cellular migration or a cascade of cytokines and/or growth factors and/or other molecules, and/or other cellular activities that are associated with tissue remodeling or wound repair.

According to the invention, various means and modalities (discussed in detail below) can be employed to provide or induce an inciting event, including, without limitation, the application of ultrasound, radio frequency (RF), laser, ultraviolet and infrared energy. Various mechanical means, such as piercing the skin and, hence, tissue can also be employed.

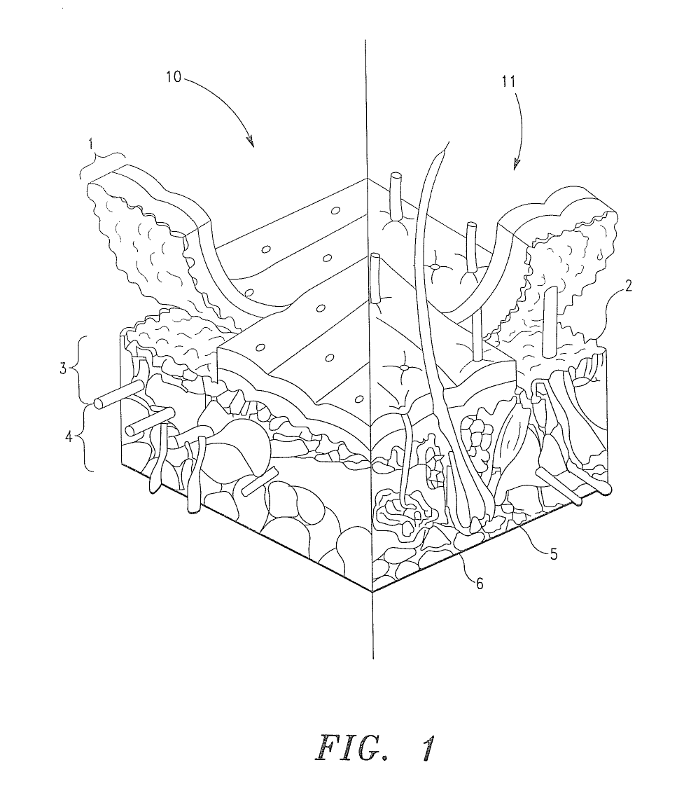

Referring now to FIG. 1, there is shown an illustration of two sections of a scalp or skin, depicting the layers therein with respect to a hairless skin layer 10 and a thin skin layer 11. As illustrated in FIG. 1, the sub-layers within the skin layers 10, 11 comprise the epidermis 12, the dermis 14, and the subcutis/hypodermis 16. The thin skin layer 11 (with hair) includes the dermal papillae 18, hair follicle 20, and eccrine sweat gland 22.

According to the invention, the depth to which an ECM composition can be administered to the scalp of a subject comprises topical and intradermal administration to the epidermis 12, intradermal administration to the dermis 14, and subcutaneous administration to the subcutis/hypodermis 16. Topical administration to the epidermis 12 corresponds to ECM composition delivery to the scalp surface, whereas intradermal administration to the epidermis 12 corresponds to a depth of approximately 50 μm up to 1.5 mm. More preferably, when the ECM composition is administered intradermally to the epidermis 12, it is delivered to a depth of approximately 100 μm up to 1 mm.

If the ECM composition is to be administered to the dermis 14, the ECM composition is preferably administered topically or to a depth of approximately 2.0 mm (0.0 mm-2.0 mm). More preferably, the ECM composition is administered at depths from approximately 0.2 mm-1.5 mm. Most preferably, the ECM composition is administered at depths from approximately 0.5 mm-1.0 mm.

According to the invention, various delivery apparatus and systems can be employed to deliver the ECM compositions to the skin of a subject and, preferably, induce an inciting event, including traditional injection means, such as needles. Other delivery means include coated microneedles, i.e. microneedles having an ECM composition deposited thereon, as well as microneedles that include internal reservoirs that are configured to receive an ECM composition therein and disperse the ECM composition therefrom.

A medicated patch or bandage can also be employed to deliver an ECM composition topically and potentially concomitantly with an injection means. This particular embodiment can be useful in situations where it is desired to deliver the ECM composition to multiple layers of the skin.

It is envisioned that the ECM composition may be used concomitantly with other procedures to achieve desired outcomes. One potential procedure is administration of a chemical peel.

In a non-limiting example, an ECM composition or graft can be administered after an alpha-hydroxy acid peel performed using glycolic acid. This kind of a chemical peel can penetrate down to the dermis and access the stem cells that allow for regeneration. In some instances it is anticipated that the chemical peel itself could contain ECM constituents that are concomitantly administered with the chemical peel.

Alternatively, the ECM composition may be injected at a depth that coincides with administration to the dermis, which after administration of the acid peel (removal of the epidermis, or otherwise alteration of normal skin layers' depth) amounts to a depth of roughly 0.5 mm-1.0 mm.

Alternatively the ECM composition may be administered as a lotion that is absorbed into the skin and helps with regeneration of the dermis after administration of the chemical peel. The removal of all or part of the epidermis via the chemical peel makes it easier for an ECM composition presented as a lotion to reach the depths necessary to incite regeneration.

In yet another non-limiting example, a phenol peel can be used to deliver an ECM composition to a very deep level. In some instances phenol peels can be used to remove precancerous growths, which, when can be used in conjunction ECM composition. In this instance the ECM composition may potentially be in a fluidized form and injected into the dermis or hypodermis as a cancer treatment, deterrent, or preventative therapy.

The different depths within the layers of skin can be reached via differing modalities. For example, micro needles could be used to administer an ECM composition to the dermal papillae. This would coincide with a depth of approximately 0.1 mm-2.0 mm. More preferably, the ECM composition is administered to a depth of 0.5 mm-1.5 mm. Also, in areas wherein cosmetic treatment is desired an incision can be made in the epidermis, wherein it is separated from the dermis, peeled back, and grafts of ECM composition administered directly to the papillary dermis beneath the epidermis, thereby creating a layer of epidermis, ECM composition, and dermal papillae; with the ECM composition being co-located with the native basement membrane.

Further examples of concomitant treatments (and inciting event inducing means) to administer with the ECM composition include electromagnetic radiation. Examples include ultrasound, radio frequency (RF), laser treatments, ultraviolet (UV), infrared, etc. In one embodiment, RF signals are administered to the desired treatment area and the ECM composition thereafter, wherein the ECM composition comprises a lotion.

In another embodiment, the ECM composition comprises an emulsion that is injected to a desired layer such as the dermis, or a desired depth, such as about 3.0 mm-4.0 mm. The RF signals are preferably administered to the desired treatment area ideally operate at frequencies of between about 30 kHz and 30 MHz. More preferably, the operational frequency ranges from about 100 kHz-1 MHz.

In another embodiment, ultrasound operating at frequencies of about 1-3 MHz is administered to the desired treatment area and thereafter an ECM composition that comprises a lotion is administered to the same region. In this embodiment, it is anticipated that added benefit may be realized if the ECM composition also comprises a topical anti-inflammatory to help accelerate the healing time post-procedure. For other electromagnetic modalities (infrared, laser, UV treatments, etc.) a similar treatment method is anticipated with respective operating frequencies found in literature.

As stated, in one embodiment of the invention, the ECM composition delivery means comprises a graft of the ECM composition. According to the invention, the ECM graft can be composed of multilayered ECM and may contain one or more of the aforementioned additives.

### EXAMPLES

The following examples are provided to enable those skilled in the art to more clearly understand and practice the present invention. They should not be considered as limiting the scope of the invention, but merely as being illustrated as representative thereof.

Patients who had undergone trauma affecting different layers of skin had their trauma site cleaned and any foreign objects removed.

The trauma sites were debrided in an effort to removal any dead or fibrotic tissue that formed in the time between when the trauma occurred and when the treatment was administered. Xenografts formed from small intestine submucosa (SIS) ECM compositions were administered to the trauma sites such that the wound was fully covered and the ECM composition was adjacent to the healthy tissue at the perimeter of the trauma site.

As the trauma site began to regenerate and replace the ECM composition with host tissue the skin reappeared from the outer perimeter towards the center of the wound. The wound reduced in size as the perimeter regenerated. In some instances, wherein a trauma site covered a large area, multiple administrations of the ECM composition were required. Secondary and tertiary administrations required that the edges be again debrided prior to readministration of the ECM composition.

The debridement and readministration of the ECM composition was repeated until the entire trauma site had been regenerated.

It was observed that as new dermis appeared, the skin appeared younger and felt softer than skin previously present, achieving often desired characterisitics associated with cosmetic procedures and compositions.

Without departing from the spirit and scope of this invention, one of ordinary skill can make various changes and modifications to the invention to adapt it to various usages and conditions. As such, these changes and modifications are properly, equitably, and intended to be, within the full range of equivalence of the following claims.

1. A composition for treating a skin disorder, comprising:
an extracellular matrix (ECM) composition, said ECM composition including an ECM material selected from the group consisting of small intestine submucosa (SIS), urinary bladder submucosa (UBS), urinary basement membrane (UBM), liver basement membrane (LBM), stomach submucosa (SS), mesothelial tissue, subcutaneous extracellular matrix, large intestine extracellular matrix, placental extracellular matrix, ornamentum extracellular matrix, heart extracellular matrix and lung extracellular matrix.

2. The composition of claim 1, wherein said ECM material comprises a decellularized ECM material.

3. The composition of claim 1, wherein said ECM material includes at least one supplemental biologically active agent.

4. The composition of claim 3, wherein said biologically active agent comprises a growth factor selected from the group consisting of a platelet derived growth factor (PDGF), epidermal growth factor (EGF), transforming growth factor-α (TGF-α), transforming growth factor-β (TGF-β), fibroblast growth factor-2 (FGF-2), basic fibroblast growth factor (bFGF), vascular epithelial growth factor (VEGF), hepatocyte growth factor (HGF), insulin-like growth factor (IGF), nerve growth factor (NGF), platelet derived growth factor (PDGF), tumor necrosis factor-α (TNA-α), and placental growth factor (PLGF).

5. The composition of claim 3, wherein said biologically active agent comprises a cell selected from the group consisting of a human embryonic stem cell, fetal cardiomyocyte, myofibroblast, mesenchymal stem cell, autotransplanted expanded cardiomyocytes, adipocyte, totipotent cell, pluripotent cell, blood stem cell, myoblast, adult stem cell, bone marrow cell, mesenchymal cell, embryonic stem cell, parenchymal cell, epithelial cell, endothelial cell, mesothelial cell, fibroblast, osteoblast, chondrocyte, exogenous cell, endogenous cell, hematopoietic stem cell, bone-marrow derived progenitor cell, myocardial cell, skeletal cell, fetal cell, undifferentiated cell, multi-potent progenitor cell, unipotent progenitor cell, monocyte, cardiac myoblast, skeletal myoblast, macrophage, capillary endothelial cell, xenogenic cell, allogenic cell and post-natal stem cell.

6. The composition of claim 3, wherein said biologically active agent comprises an active agent selected from the group consisting of a collagen (types I-V), proteoglycans, glycosaminoglycans (GAGs), glycoproteins, cytokines, cell-surface associated proteins, cell adhesion molecules (CAM), endothelial ligands, matrikines, cadherins, immuoglobins, fibril collagens, non-fibrallar collagens, basement membrane collagens, multiplexins, small-leucine rich proteoglycans, decorins, biglycans, fibromodulins, keratocans, lumicans, epiphycans, heparin sulfate proteoglycans, perlecans, agrins, testicans, syndecans, glypicans, serglycins, selectins, lecticans, aggrecans, versicans, neurocans, brevicans, cytoplasmic domain-44 (CD-44), macrophage stimulating factors, amyloid precursor proteins, heparins, chondroitin sulfate B (dermatan sulfate), chondroitin sulfate A, heparin sulfates, hyaluronic acids, fibronectins, tenascins, elastins, fibrillins, laminins, nidogen/enactins, fibulin I, finulin II, integrins, transmembrane molecules, thrombospondins, ostepontins, and angiotensin converting enzymes (ACE).

7. The composition of claim 3, wherein said biologically active agent comprises a pharmacological agent.

8. The composition of claim 7, wherein said pharmacological agent is selected from the group consisting of antibiotics, antifungal agents, anti-viral agents, anti-pain agents, anesthetics, analgesics, steroidal anti-inflammatories, non-steroidal anti-inflammatories, anti-neoplastics, anti-spasmodics, modulators of cell-extracellular matrix interactions, proteins, hormones, enzymes and enzyme inhibitors, anticoagulants, antithrombic agents, DNA, RNA, modified DNA and RNA, NSAIDs, inhibitors of DNA, polypeptides, oligonucleotides, polynucleotides, nucleoproteins, and vasodilating agents.

9. The composition of claim 7, wherein said pharmacological agent comprises a HMG-CoA reductase inhibitor.

10. The composition of claim 9, wherein said HMG-CoA reductase inhibitor is selected from the group consisting of atorvastatin, cerivastatin, fluvastatin, lovastatin, mevastatin, pitavastatin, pravastatin, rosuvastatin and simvastatin.

11. A method of treating a skin disorder of a subject, comprising the steps of:
providing an ECM composition including at least one ECM material selected from the group consisting of small intestine submucosa (SIS), urinary bladder submucosa (UBS), urinary basement membrane (UBM), liver basement membrane (LBM), stomach submucosa (SS), mesothelial tissue, subcutaneous extracellular matrix, large intestine extracellular matrix, placental extracellular matrix, ornamentum extracellular matrix, heart extracellular matrix and lung extracellular matrix;
administering inciting event means to a target skin location on the subject to induce at least one inciting event at said target skin location; and
administering a therapeutically effective amount of said ECM composition to said target skin location.

12. The method of claim 11, wherein said ECM material comprises a decellularized ECM material.

13. The method of claim 11, wherein said ECM material includes at least one additional biologically active agent.

14. The method of claim 13, wherein said biologically active agent comprises a growth factor selected from the group consisting of a platelet derived growth factor (PDGF), epidermal growth factor (EGF), transforming growth factor-α (TGF-α), transforming growth factor-β (TGF-β), fibroblast growth factor-2 (FGF-2), basic fibroblast growth factor (bFGF), and vascular epithelial growth factor (VEGF).

15. The method of claim 13, wherein said biologically active agent comprises a human embryonic stem cell.

16. The method of claim 13, wherein said biologically active agent comprises an active agent selected from the group consisting of proteoglycans, glycosaminoglycans (GAGs), glycoproteins, and cytokines.

17. The method of claim 13, wherein said biologically active agent comprises a pharmacological agent selected from the group consisting of steroidal anti-inflammatories and non-steroidal anti-inflammatories.

18. The method of claim 17, wherein said pharmacological agent comprises a HMG-CoA reductase inhibitor selected from the group consisting of atorvastatin, cerivastatin, fluvastatin, lovastatin, mevastatin, pitavastatin, pravastatin, rosuvastatin and simvastatin.

19. The method of claim 11, wherein said inciting event means comprises the administration of energy to said target skin location of the subject, said inciting event energy being selected from the group consisting of ultrasonic energy, radio frequency energy, laser energy, ultraviolet energy and infrared energy.
